(19) United States
(12) Patent Application Publication
(10) Pub. No.: US 2024/0417713 A1
(43) Pub. Date: Dec. 19, 2024

(54) VIRUS TO EXPRESS GAD65 UNDER E2 REGULATORY ELEMENT

(71) Applicant: New York University, New York, NY (US)

(72) Inventors: Dan H. Sanes, New York, NY (US); Samer Masri, Brooklyn, NY (US)

(21) Appl. No.: 18/741,354

(22) Filed: Jun. 12, 2024

Related U.S. Application Data
(60) Provisional application No. 63/507,894, filed on Jun. 13, 2023.

(51) Int. Cl. C12N 9/88; A61K 48/00; A61P 27/16; C12N 15/86

(52) U.S. Cl. CPC C12N 9/88; A61K 48/0058; A61P 27/16; C12N 15/86; C12Y 401/01015; C12N 2710/10043

(57) ABSTRACT

Provided are polynucleotides and viral vectors encoding glutamate decarboxylase (GAD65) under control of the E2 regulatory element. The sequence encoding the GAD65 is operably linked to a genetic element that can selectively drive expression of the GAD65 protein in parvalbumin-positive interneurons (PV) neurons, illustrated using a S5E2 element. GAD65 expression results in restored spectral modulation (SM) detection following developmental hearing loss and increases transmission of GABA at both GABA_{A} and GABA_{B} receptors.

### CROSS REFERENCE TO RELATED APPLICATIONS

This application claims the benefit of U.S. provisional patent application No. 63/507,894, filed Jun. 13, 2023, the entire disclosure of which is incorporated herein by reference.

### STATEMENT REGARDING FEDERALLY SPONSORED RESEARCH

This invention was made with government support under R01 DC011284 awarded by the National Institutes of Health. The government has certain rights in the invention

### FIELD

The present disclosure relates generally to prophylaxis and/or therapy of hearing loss, and more specifically to use of recombinant polynucleotides and viral vectors with a sequence encoding GAD65 protein under the control of a genetic element to restrict its expression to certain types of neurons.

### BACKGROUND OF THE DISCLOSURE

Reduced cortical inhibition has been implicated in a broad range of developmental disorders including epilepsy, autism, schizophrenia, fragile x syndrome, and impaired sensory processing (Sanes & Kotak, 2011; Braat & Kooy, 2015; Gainey & Feldman, 2017). For example, visual or auditory deprivation that occurs during developmental sensitive periods lead to weaker inhibitory synapses between GABAergic interneurons and pyramidal cells in primary sensory cortices (Maffei et al. 2004; Takesian et al. 2012; Mowery et al., 2015). In some cases, these functional effects are correlated with a down-regulation of GABA receptors or loss of GABAergic terminals (Fuchs & Salazar, 1998; Kilman et al. 2002; Jiao et al. 2006; Sarro et al. 2008; Braat et al. 2015). Furthermore, when induced by hearing loss (HL), this reduction of inhibition has been linked to a broad range of perceptual and central processing deficits (Aizawa and Eggermont, 2007, Rosen et al., 2012; Yao and Sanes, 2018; Gay et al., 2014; Polley et al., 2013; Han et al., 2007; Kim and Bao, 2009; Zhang et al., 2001; Mowery et al., 2019). Taken together, these observations lead to the hypothesis that developmental HL induces a reduction of postsynaptic GABA receptor-mediated inhibition in auditory cortex (AC), thereby causing perceptual deficits. There is indirect support for the premise that normal perceptual performance is associated with appropriate levels of cortical inhibition in adults. For example, magnetic resonance spectroscopy measurements in humans demonstrate that performance on visual or auditory perceptual tasks are correlated with a higher GABA concentration (Edden et al., 2009; Dobri and Ross, 2021; Ip et al., 2021). Furthermore, a pharmacological manipulation that enhances GABAergic inhibition during behavioral testing leads to improved performance on an auditory temporal perception task in senescent gerbils and improved visual coding in senescent monkeys (Gleich et al., 2003; Leventhal et al. 2003). Consistent with this idea, systemic treatment with a GABA reuptake inhibitor can both restore the strength of inhibitory synapses following developmental HL and rescue an auditory perceptual skill (Kotak et al., 2013; Mowery et al., 2019). Although the relationship between inhibition and mature sensory processing is well established, the relative contribution of ionotropic GABA_{A} and metabotropic GABAB postsynaptic receptors is uncertain. Depending on the outcome measure, pharmacological experiments suggest that both types of receptors can be an effective target for restoring normal function or plasticity (Iwai et al. 2003, Möhler et al., 2004; Fagiolini et al., 2004; Kotak et al., 2013; Cai et al., 2017). Therefore, selective gain-of-function manipulations are required to determine whether restoring GABA_{A} or GABA_{B} receptor-mediated inhibition can remediate a behavioral deficit resulting from a developmental insult. There is accordingly an ongoing and unmet need for improved compositions and methods that relate to hearing loss and other disorders in a manner that relates to GABA_{A} or GABA_{B} receptor-mediated inhibition. The present disclosure is pertinent to this need.

### BRIEF SUMMARY OF THE DISCLOSURE

The present disclosure relates to compositions and methods for use in prophylaxis and/or therapy of hearing loss-induced deficits, developmental disorders, and disorders of inhibition. Aspects of the disclosure relate in part to the demonstration that upregulating GABA synthesis in parvalbumin-positive interneurons (PV) neurons) is sufficient to improve auditory perceptual skills that are associated with hearing loss.

The disclosure provides polynucleotides encoding glutamate decarboxylase (GAD65) under control of the E2 regulatory element. The sequence encoding the GAD65 protein may be provided in the form of a viral vector, illustrated using a recombinant adeno associated virus (rAVV) vector. The sequence encoding the GAD65 protein is operably linked to a genetic element that can selectively drive expression of the GAD65 protein in PV neurons, illustrated using a S5E2 element, which is referred to the art as a promoter or enhancer or regulatory element. In embodiments, the described GAD65 expression results in restored spectral modulation (SM) detection following developmental hearing loss. In embodiments, the described expression of GAD65 will increase transmission of GABA at both GABA_{A} and GABA_{B} receptors.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1: Viral vector design and validation. (Panel A) For both Gabra1 and Gabbr1b AAVs, primary auditory cortex layer 2/3 was injected. After three weeks a thalamocortical slice preparation was made and whole cell recordings (current clamp) from ACx L2/3 pyramidal cells were carried out. (Panel B) Top, Diagram showing Gabra1 vector. Bottom, micrograph from ACx L2/3 showing Gabra1 infected cells (mCherry) and one patched pyramidal neuron. (Panel C) Representative evoked IPSPs showing the larger GABA_{A} potential in the Gabra1 infected pyramidal neuron (fluorescing patched cell from Panel B) vs local uninfected (non-fluorescing) pyramidal neuron from the same slice. (Panel D) Plot diagram showing the difference in GABA_{A} IPSP amplitudes for uninfected versus Gabra1 infected pyramidal neurons in adults. (Panel E) Top, Diagram showing Gabbr1b vector. Bottom, micrograph from AC L2/3 showing Gabbr1b infected cells (turboRFP) and one patched pyramidal neuron. (Panel F) Representative evoked IPSPs showing the larger GABA_{B} potential in the Gabbr1b infected pyramidal neuron (from Panel E) vs. local uninfected (non-fluorescing) pyramidal neuron from the same slice, in the presence of bicuculline to block GABA_{A} receptors. (Panel G) Plot diagram showing the difference in GABA_{B} IPSP amplitudes for uninfected versus Gabbr1b infected pyramidal neurons in adults. (Panel H) Micrograph from AC L2/3 showing Gabbr1b infected cells and one patched pyramidal neuron (Panel I) Representative evoked IPSPs showing the larger GABA_{B} potential in the Gabbr1b infected pyramidal neuron (from Panel H) vs. a local uninfected (non-fluorescing) pyramidal neuron from the same slice in a P45 animal. Bicuculline was added to block GABA_{A} receptors. (Panel J) Plot diagram showing the difference in GABA_{B} IPSP amplitudes for uninfected versus Gabbr1b infected pyramidal neurons in P45 animals.

FIG. 2: Experimental paradigm. (Panel A) The experimental timeline, and each of the experimental groups is shown. (Panel B) Example stimulus waveforms are shown for the AM depth detection task (top) and the SM depth detection task (bottom). (Panel C) The Go-Nogo paradigm used for psychometric testing is shown.

FIG. 3: Gabbr1b expression restores AM detection. Gabbr1b expression restores AM detection. (Panel A) Representative behavior for a HL-reared gerbil expressing Gabbr1b (HL+Gabbr1b) and a HL-reared gerbil expressing GFP (HL+GFP) in AC, both tested after transient hearing loss (HL). (Panel B) AM depth thresholds achieved by each group over training days. Mean±SEM. (Panel C) Gabbr1b expression in AC rescued AM perception relative to GFP expression on day 1 of psychometric testing (bars with solid outlines). Gabbr1b expression in AC rescued AM perception relative to GFP expression on day 7 of psychometric testing (bars with dashed outlines). Gray lines show changes in threshold for each animal from day 1 to 7 of testing. Significant differences indicated by asterisks (see text for statistical values).

FIG. 4. Gabbr1b and GAD65 expression restore SM detection. (FIG. 4 panel A) Example psychometric curves of individuals showing d′ at each of the 5 modulation depths presented in a single session. The leftward shift of the HL+Gabbr1b function, relative to HL+GFP function indicates improved performance. Bars indicate significant differences (see text for statistical values). (FIG. 4, panel B) Group performance on each day of psychometric testing. (FIG. 4, panel C) SM thresholds on day 1 (solid bars) and 7 (dashed bars) of psychometric testing. There are no differences in SM modulation thresholds on the first day of psychometric testing, as calculated by psychometric fit crossing d′=1. HL+Gabbr1b, HL+GAD65 and NH+GFP groups performed significantly better than HL+GFP animals. Gray lines show changes in threshold for each animal from day 1 to 7 of testing. Significant differences indicated by asterisks (see text for statistical values).

FIG. 5: Spectral modulation detection in normal hearing juvenile gerbils. (Panel A) NH animals display better thresholds for SM at 2 cycles/octave relative to 10. Bar indicates significant difference (see text for statistical value). (Panel B) SM detection thresholds do not change significantly when sound is presented at a lower level of 36 dB SPL (p=0.593).

FIG. 6: Procedural training for spectral modulation detection. (Panel A) There are no group differences in improvement rate for procedural training (mean±SEM, moving window of 20 trials). (Panel B) There are no significant differences in maximum d′ achieved during all procedural training over 20 trial windows (F=2.439, df=32, p=0.0703).

FIG. 7: Colocalization of parvalbumin antibody and fluorescent reporter. (Panel A) Image of auditory cortex showing fluorescent reporter (turboRFP) expressed under the S5E2 enhancer element in a AAV-GAD65 injected animal. (Panel B) Pseudo-green signal of Alexa 647 secondary antibody after primary antibody staining for parvalbumin. In all images, arrows indicate neurons that only exhibit PV staining, and do not express turboRFP. (Panel C) Merged image. (Panels D-F) Higher power images of panels A-C (area within white dashed rectangle).

FIG. 8: Gabbr1b expression in normal hearing animals impairs SM detection (Panel A) Group performance on each day of psychometric testing. Panel (B) SM thresholds on day 1 (solid bars) and 7 (dashed bars) of psychometric testing. Gray lines show changes per animal. Star indicates p=0.021.

### DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide and amino acid sequences described herein, and every polynucleotide sequence referred to herein includes its complementary DNA sequence, and also includes the RNA equivalents thereof to the extent an RNA sequence is not given. Every DNA and RNA sequence encoding polypeptides disclosed herein is encompassed by this disclosure, including but not limited to sequences encoding all recombinant proteins that comprise a segment of or a full protein, as described further below. Any sequence referred to by a database entry is incorporated herein by reference as the sequence exists in the database as of the effective filing date of this application or patent.

The present disclosure comprises compositions and method for prophylaxis and/or therapy of disorders which attend focal or systemically reduced inhibition in the brain. These include but are not limited to epilepsy, autism, fragile x, and schizophrenia. These also include sensory disorders such as hearing loss or related dysfunctions, including but not limited to tinnitus, that could be ameliorated by restoring central nervous system inhibitory synapses. In embodiments, the individual has amblyopia, developmental cataracts, or another disorder related to sensory perception. Thus, in embodiments, the disclosure relates to prophylaxis and/or therapy of central nervous system sequelae of hearing loss. The disclosure provides recombinant polynucleotides and viral vectors that are used to express at GAD65 under the control of an S5E2 element.

Certain examples and reductions to practice described herein were developed using the described gerbil models. Amino acid and nucleotide sequences of the described viral constructs that were used to produce the results are provided below. Based on the analysis using gerbils, it is expected that the same approach can be used with other mammals, including but not necessarily limited to humans.

The construct used to express the GAD65 protein is configured such that its expression is driven by a genetic element referred to in the art as the “S5E2” that is operably linked to the GAD65 protein coding sequence. By “operably linked” it is meant that the S5E2 sequence is present in the same polynucleotide as the sequence encoding the GAD65 protein (and is thus provided in cis), and expression of the GAD65 protein is dependent on the presence and function of the promoter to promote transcription in a manner that is restricted to parvalbumin-positive interneurons. The sequence of the S5E2 genetic element is known in the art, such as by way of U.S. patent publication no. 20220195457, from which the description and sequences of the S5E2 genetic element is incorporated herein by reference. Likewise, the sequence of human GAD65 is known in the art, such as from GenBank accession no. OP680451.1, from which the amino acid sequence of the GAD65 sequence is incorporated herein by reference as it exists in the database as of the filing date of this application. In an example, the sequence of GAD65 used in embodiments of this disclosure is at least 80% identical across the length of the human GAD65 amino acid sequence. In examples, the sequence of GAD65 used in embodiments of this disclosure is at least 90, 91, 92, 93, 94, 95, 96, 97, 98, 99% identical across the length of the human GAD65 amino acid sequence.

In embodiments, a viral vector is used to introduce the described polynucleotides into an individual. In an embodiment, the viral vector comprises a retroviral vector, such as a lentiviral vector. In embodiments, the viral vector comprises a recombinant adeno-associated virus (rAAV). In one embodiment, the viral vector comprised a self-complementary adeno-associated virus (scAAV).

Methods of this disclosure comprise introducing the modified rAAVs into neuronal cells in the brain of an individual in need thereof. Non-limiting embodiments of this disclosure are demonstrated in gerbils to demonstrate effects on sound perception. However, and as will be recognized by those skilled in the art, because the present disclosure includes a method for modifying neurons so that they comprise the described features, it is feasible for the present disclosure to have additional therapeutic applications that extend beyond hearing loss.

Suitable vectors that can be adapted to comprise a suitable promoter and encode the GAD65 component, given the benefit of the present disclosure, are commercially available from, for example, the CLONTECH division of TAKARA BIO. In certain implementations plasmid vectors may encode all or some of the well-known rep, cap and adeno-helper components. The rep component comprises four overlapping genes encoding Rep proteins required for the AAV life cycle (Rep78, Rep68, Rep52 and Rep40). The cap component comprises overlapping nucleotide sequences of capsid proteins VP1, VP2 and VP3, which interact together to form a capsid of an icosahedral symmetry. Another plasmid providing the Adeno Helper function may also be co-transfected. The helper components comprise the adenoviral genes E2A, E4orf6, and VA RNAs for viral replication.

In embodiments, the polynucleotides encoding the GAD65 protein in the rAAV can be modified, for example, by including optimized codons for expression in human neurons.

The described polynucleotides and vectors comprising the polynucleotides encode the GAD65 protein and comprise a promoter that is operably linked to the GAD65 coding sequence. Other optional features of the vectors are illustrated in the accompanying figures and the description below. In one embodiment, such as for therapeutic purposes, the polynucleotide can be free from any sequence encoding a reporter protein. Likewise, components of the polynucleotides that are included for the purpose of expressing a reporter protein and visualizing the location of expression may be excluded from polynucleotides that are intended for therapeutic approaches. However, polynucleotides of this disclosure can comprise additional elements that will be apparent to those skilled in the art, given the benefit of the present disclosure.

In certain examples, the polynucleotides comprise a sequence encoding an element such as a Woodchuck hepatitis virus Posttrascriptional Regulatory Element (WPRE) or a variant thereof, which is believed to increase RNA stability and protein yield. The polynucleotides may also comprise a polyadenylation signal such as bovine growth hormone polyadenylation signal and/or SV40 polyomavirus simian virus 40 polyadenylation signal. The polynucleotide can comprise a minimal promoter, such as a human beta-globin minimal promoter (phβg) and a chimeric intron sequence. Without intending to be constrained by any particular theory it is considered that described rAVV vectors aid in concatamer formation in the nucleus after the single-stranded vector DNA is converted by host cell DNA polymerase complexes into double-stranded DNA. It is accordingly believed that administration of the rAAVs of this disclosure will form episomal concatemers in the nucleus of cells into which they are introduced. In non-dividing cells, such as adult neurons, it is believed these concatemers remain intact for the life of the neurons. It is also expected that integration of rAAV polynucleotides into host chromosomes will be negligible or absent and will not affect expression of regulation of any other human gene.

In various embodiments, the disclosure includes isolated and/or recombinant polynucleotides comprising a described genetic element and the sequence encoding the GAD65 protein, expression vectors comprising such polynucleotides, cells comprising the polynucleotides, cells comprising rAAV encoded by the polynucleotides, isolated preparations of such rAAV particles, and pharmaceutical preparations comprising the rAAV particles.

In various aspects of the invention, methods of making the rAAVs are provided. In general, the method of making the rAAvs comprises culturing cells which comprise an expression vector encoding an rAAV of this disclosure, allowing expression of the polynucleotides to produce the rAAVs, and separating the rAAVs from cells in the cell culture and/or from the cell culture media. The rAAVs can be purified to any desired degree of purity using conventional approaches.

rAAVs of the invention can be mixed with any pharmaceutically acceptable buffer, excipient, carrier and the like to form a pharmaceutical preparation. Suitable pharmaceutical compositions can be prepared by mixing rAAVs with a pharmaceutically-acceptable carrier, diluent or excipient, and suitable such components are well known in the art. Some examples of such carriers, diluents and excipients can be found in: Remington: The Science and Practice of Pharmacy (2022) 23rd Edition, Philadelphia, PA.

In general, a composition comprising a rAAV can be administered to any individual in need thereof. In embodiments, the individual is suffering from hearing loss, has suffered hearing loss, or is at risk for progression of hearing loss. In embodiments, the individual has been diagnosed with or is suspected of having peripheral hearing loss (i.e., either temporary or permanent dysfunction of the middle ear bones or the cochlea). In embodiments, the individual is suffering from an analogous sensory disorder, such as cataracts or amblyopia.

The rAAV can be administered using any suitable approach, such as intracranial injection or intravenous injections. In embodiments, the described rAAVs can be administered such that they enter and express the GAD65 protein components at least in parvalbumin-positive interneurons, or only in parvalbumin-positive interneurons.

In embodiments, the disclosure includes administering a therapeutically effective amount of an rAAV to an individual. “Therapeutically effective amount” as used herein means that amount of rAVV that is introduced into a sufficient number of neurons such that hearing loss-induced deficits or other disorders are inhibited or reversed. The amount of rAAV that is administered can be determined by those skilled in the art, given the benefit of the present disclosure and based on factors such as the size of the individual, age, gender, type, and severity of hearing loss. In embodiments, a therapeutically effective amount is an amount sufficient such that the sequelae of hearing loss in the individual is inhibited, or hearing of the individual is improved. In embodiments, a therapeutically effective amount is sufficient such that degenerative changes within the central nervous system that are induced by peripheral hearing loss are inhibited or prevented. In an embodiment, inhibiting one central sequela of hearing loss that comprises a reduction of auditory cortex synaptic inhibition mediated by the production of GABA. In an embodiment, the disclosure provides for recovery of normal performance on auditory perceptual tasks, non-limiting examples of which are described below. In embodiments, comprehension of human speech is improved.

The approaches of the present disclosure can also be combined with other anti-hearing loss techniques, including but not necessarily limited to use with therapeutic agents, and/or medical devices.

Without intending to be bound by any particular interpretation, the virally-mediated protein expression of this disclosure has at least the following advantages: The expression of the GAD65 protein is localized to a small volume around the injection location, and can thus be targeted to any brain region exhibiting reduced inhibition, such as an epileptogenic focus. The expression of the GAD65 protein receptor can be further restricted to specific types of neurons in the injection site through the use of a specific gene promotor, illustrated with the S5E2 enhancer.

The Examples below are intended to illustrate but not limit aspects of this disclosure. The Examples include discussion of increasing the functional expression of either GABA_{A} or GABA_{B}-mediated synaptic inhibition in the auditory cortex. These embodiments are included in the present disclosure, but germane to the foregoing description and embodiments of this disclosure are the examples that demonstrate upregulating GABA synthesis in parvalbumin-positive interneurons with a virus that expressed the 65-kD isoform of glutamate decarboxylase (GAD65) under the S5E2 enhancer (Vormstein-Schneider et al. 2020). The approach employed a previously validated paradigm in which transient developmental hearing loss (HL) is induced during the AC critical period, causing a reduction in GABA_{A} and GABA_{B}-mediated AC inhibition and diminished performance on an amplitude modulation (AM) detection task (Mowery et al., 2015; Caras and Sanes, 2015; Mowery et al., 2016; Mowery et al., 2019). We also introduce a second perceptual task, spectral modulation (SM) detection, with which to assess the effect of HL and the effect of restoring inhibition. The ability to perceive spectral modulation of sound is especially important for speech and speech-in-noise comprehension (Drullman, 1995; Zeng et al. 2005). Furthermore, humans with hearing loss or cochlear implants are impaired in spectral modulation detection, and this is correlated with speech perception (Horn et al. 2017; Ozmeral et al., 2018; Nittrouer et al., 2021). Together, AM and SM cues compose two of the fundamental building blocks of natural sounds (Singh and Theunessin, 2003), and HL-induced deficits have been linked to delayed speech and language acquisition. The disclosure illustrates that viral expression of a GABA_{B} receptor subunit, but not a GABA_{A} receptor subunit, in the AC can remediate the deleterious effects of developmental HL on auditory perception, and also demonstrate that increased GAD65 protein expression has similar effects.

The following Examples are intended to illustrate aspects of the disclosure but are not intended to be limiting.

### Example 1

### Experimental Procedures

Experimental animals: We performed behavioral experiments on 52 normal hearing and 47 transient HL gerbils (Meriones unguiculatus) in the age range of postnatal days (P) 10-48. For brain slice experiments, we recorded from 34 AC pyramidal neurons, obtained from a total of 8 male and female gerbils in the age range P103-169. All animals were born from breeding pairs (Charles River Laboratories) in our colony. All procedures were approved by the Institutional Animal Care and Use Committee at New York University.

Induction of transient hearing loss: Reversible HL was induced using earplugs made of molding clay inserted in both ears after ear canal opening, at P10, and sealed with super glue (Mowery et al., 2015; Mowery et al., 2016). Earplugs were checked daily, replaced if needed, and removed at P23. This manipulation produces a threshold shift of 15-50 dB, depending on frequency, as measured with auditory brainstem responses (Caras and Sanes, 2015), and ˜25 dB at 4 kHz as measured behaviorally (Mowery et al., 2015).

Auditory cortex brain slice recordings: Thalamocortical brain slices were generated as described previously (Kotak et al., 2005; Mowery et al., 2015, 2019). Animals were deeply anesthetized (chloral hydrate, 400 mg/kg, IP) and brains dissected into 4° C. oxygenated artificial cerebrospinal fluid (ACSF, in mM: 125 NaCl, 4 KCl, 1.2 KH2PO4, 1.3 MgSO4, 24 NaHCO₃, 15 glucose, 2.4 CaCl2, and 0.4 L-ascorbic acid; and bubbled with 95% 02-5% CO2 to a pH=7.4). Brains were vibratome-sectioned to obtain 300-400 μm perihorizontal auditory thalamocortical slices. The AC was identified by extracellular field responses to medial geniculate stimulation.

Whole-cell current clamp recordings were obtained (Warner PC-501A) from AC layer 2/3 pyramidal neurons at 32° C. in oxygenated ACSF. Recording electrodes were fabricated from borosilicate glass (1.5 mm OD; Sutter P-97). The internal recording solution contained (in mM): 5 KCl, 127.5 K-gluconate, 10 HEPES, 2 MgCl2, 0.6 EGTA, 2 ATP, 0.3 GTP, and 5 phosphocreatine (pH 7.2 with KOH). The resistance of patch electrodes filled with internal solution was between 5-10 MQ2. Access resistance was 15-30 MQ2, and was compensated by about 70%. Recordings were digitized at 10 kHz and analyzed offline using custom Igor-based macros (IGOR, WaveMetrics, Lake Oswego, OR). All recorded neurons had a resting potential≤−50 mV and overshooting action potentials.

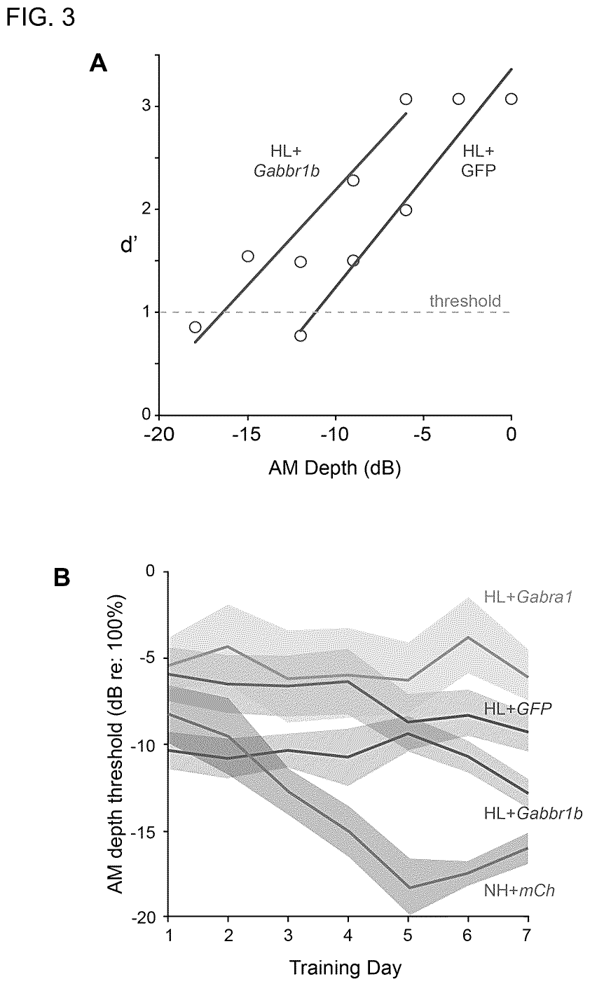

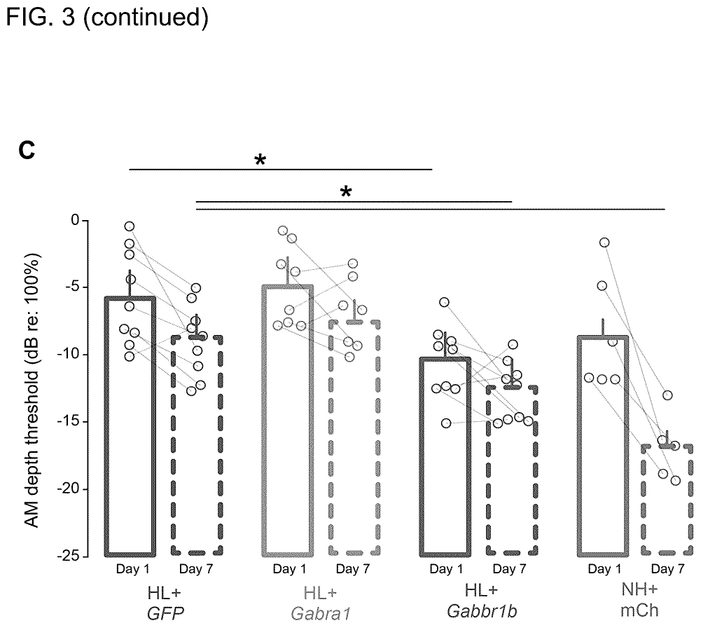

Inhibitory postsynaptic potentials (IPSPs) were evoked via biphasic stimulation of layer 4 (1-10 mV, 10 s interstimulus interval) in the presence of ionotropic glutamate receptor antagonists (6,7-Dinitroquinoxaline-2,3-dione, DNQX, 20 μM; 2-amino-5-phosphonopentanoate, AP-5, 50 μM). The drugs were applied for a minimum of 8 min before recording IPSPs. Peak amplitudes of the short latency hyperpolarization (putative GABA_{A} component) and long latency hyperpolarization (putative GABA_{B} component) were measured from each response at a holding potential (Vhold) of −50 mV. To assess GABA_{B} receptor mediated IPSPs, the GABA_{A} receptor antagonist bicuculline (10 μM) was also added to the bath. We previously verified that short- and long-latency IPSP components represented GABA_{A} and GABA_{B} receptor-dependent responses, respectively (see FIG. 3D in Mowery et al., 2019).

Behavioral training and testing: Amplitude modulation (AM) and spectral modulation (SM) depth detection thresholds were assessed with an aversive conditioning procedure (Heffner & Heffner, 1995) as previously described (Sarro & Sanes, 2011; Rosen et al., 2012; Buran et al., 2014; Caras and Sanes, 2015, 2017, 2019). The apparatus was controlled by custom Matlab scripts, interfaced with a digital signal processor (TDT). Stimuli were delivered via a calibrated tweeter (KEF electronics) positioned above a test cage which contains a metal water spout and floor plate. Water delivery was initiated by a syringe pump (Yale Apparatus) triggered by infrared detection of spout contact. The speaker and cage were located in a sound attenuation chamber and observed via video monitor. After placement on controlled water access, gerbils learned to drink steadily from the lick spout while in the presence of continuous, unmodulated, band-limited noise (0.1-20 kHz). Separate groups of animals were trained to withdraw from the spout when the sound changed from unmodulated noise (the “safe” cue) to either AM or SM noise (the “warn” cue) by pairing the modulation with a mild shock (0.5-1.0 mA, 300 ms; Lafayette Instruments) delivered through the spout. For the AM task, procedural training was conducted with a warn cue of 0 dB re: 100% modulation depth. For the SM task, procedural training was conducted with a warn cue of 40 dB modulation depth. Repeated pairings of the shock and the warn cue resulted in a rapidly learned association and reliable spout withdrawal, which was used as a behavioral measure of modulation detection. Warn trials were interspersed with 2-6 safe trials (2-6 seconds), during which the unmodulated sound continued unchanged; the unpredictable nature of the warn presentation prevented temporal conditioning.

After the initial associative learning, five AM or SM depths, bracketing the threshold (AM task: −3 to −27 dB re: 100% depth in 3 dB steps; SM task: +3 to 27 dB), were presented in descending order. Note that AM depth was calculated relative to a completely modulated sinusoidal waveform (dB re: 100% depth), such that larger negative values represent depths that are more difficult to detect. SM depth was calculated relative to unmodulated noise, such that smaller positive values represent depths that are more difficult to detect (dB re: 0% depth). Average stimulus level was held constant at 45 dB SPL to ensure that detection was based on the modulation cue. SM stimuli were generated using 3200 random-phase sinusoidal components spaced between 0.1 and 20 kHz at a sampling rate of 44.8 Khz (code courtesy of Dr. Donal Sinex). Stimuli were 1 second long with a 20 ms on and off ramps. Stimuli were generated at 2 or 10 cycles/octave. For experiments testing the effects of HL and viral vectors, SM density (2 cycles/octave) and AM rate (5 Hz) remained constant. Psychometric testing spanned 7 or 10 consecutive days. On warn trials, the response was scored as a hit when animals withdrew from the spout. On safe trials, the response was scored as a false alarm when animals incorrectly withdrew from the spout. These responses were used to calculate d′as z(hits)−z(false alarms), a signal detection metric that accounts for individual guessing rates (Green, 1966). Values were fit with psychometric functions and used to calculate thresholds (Wichmann & Hill, 2001a, 2001b). Threshold was defined as the smallest stimulus depth at which d′=1.

Development of viral vectors: Two viral vectors were developed to express either the postsynaptic GABA_{B} 1b subunit (Billinton et al., 1999), or the GABA_{A}α1 subunit under the CaMKII promoter. The gene sequences for each (Gabbr1b and Gabra1) were extracted from the gerbil genome using BLAST (Zorio et al., 2019). For the Gabbr1b sequence, results were missing the first 141 bp of the full gene sequence, which were replaced with a sequence from the mouse to generate a complete gene sequence. These genes were inserted into viral cassettes with fluorescent reporters. At 2532 base pairs, the Gabbr1b sequence was too long to be used in the same cassette used for the GABA_{A} subunit while maintaining high expression levels in an AAV1 serotype. We therefore minimized cassette length by using TurboRFP as the fluorescent reporter, WPRE3 as a posttranscriptional regulatory element, and P2A to cleave the fluorescent reporter (Merzlyak et al., 2007; Choi et al., 2014, Zufferey et al., 1999; Szymczak et al., 2005). A third vector was developed using this same cassette, expressing GAD65 under the S5E2 promotor (Vormstein-Schneider et al. 2020). Genes were synthesized (Gabra1: ThermoFisher; Gabbr1b: Genewiz), and the full plasmids were generated, cloned, and packaged (Penn Vector Core): AAV1.CaMKII0.4.Gabbr1b.P2A. TurboRFP.WPRE3.rBG (4×10¹² viral particles/mL), AAV1.CaMKII0.4.Gabra1.IRES.mCherry.WPRE.rBG (7×10¹² viral particles/mL) and AAV1.S5E2.GAD65.P2A.TurboRFP.WPRE3.rBG (7×10¹² viral particles/mL).

Virus injections: For all animals, bilateral surgical virus injections into the AC were performed on P23 or P24, after earplug removal. AAV1.hSyn.eGFP.WPRE.bG (5×10¹² vg/mL) was used as a control. Animals were anesthetized with isoflurane, and incisions made just ventral to the temporal ridge. A small burr hole (0.7 mm diameter) was drilled in the skull˜1.4 mm below the temporal ridge, just over the AC (Radtke-Schuller et al., 2016). Virus was loaded into a glass pipette backfilled with mineral oil. The glass pipettes were made sharp enough to penetrate dura without causing damage and were inserted to a depth of 400 μm. Virus was injected at a rate of 2 nl/s with a Nanoject III (Drummond), followed by a 5 min period to permit diffusion. For the two custom viruses, a volume of 600 μl was injected, whereas for the control GFP virus, a volume of 250 μl was injected. Incisions were closed with surgical adhesive and the animal permitted to recover for 7 days.

Histology: At the end of each experiment, animals were deeply anesthetized with Euthasol and perfused transcardially with ice-cold PBS followed by 4% paraformaldehyde. Brains were removed and fixed in the same fixative overnight at 4° C. Brains were cryoprotected and sectioned (70 μm) on a vibratome and processed for fluorescence imaging. To confirm targeting of the S5E2 enhancer element we incubated free floating brain sections 2 weeks after injection in a solution containing anti-Parvalbumin antibody (MAB1572, Sigma-Aldrich) at a concentration of 1:1000 and triton (0.2%) for 24 hours at room temperature. After rinsing, sections were transferred to a solution with secondary antibodies (Goat anti-Mouse AlexaFluor 647, A-21235, Thermofisher) at a concentration of 1:200 for 2 hours. Sections were mounted and assessed with epifluorescence (ECHO Revolve) and confocal imaging.

Statistical analysis. When data were normally distributed (as assessed by the Shapiro-Wilk W Test), values were given as mean±SEM. Statistical analyses were conducted using MatLab. To compare multiple measures obtained from the same animal, a linear mixed effects model, correcting for subject identity (formula: threshold˜1+group*training day+(1|subject)) was used to verify a main effect of treatment group or the interaction between training day and treatment group, For individual training days, group comparisons of variables were made using a one-way analysis of variance (ANOVA) followed by least significant differences post-hoc. The HL+GFP group was specified as the control group for all post hoc tests. The best threshold achieved by each animal during 7 or 10 days of psychometric testing (depending on the experiment) was used to determine perceptual thresholds. The significance level was set at α=5%. All data are expressed as mean±SEM unless otherwise stated.

### Example 2

Example 2 and the discussion that follows it provide a description of results obtained using the materials and methods described in Example 1.

To investigate the causal relationship between AC synaptic inhibition and HL-induced perceptual deficits, the present disclosure provides viral vectors to express the gerbil gene sequences for: subunit al of the GABA_{A} receptor (Gabra1) or subunit 1b of the GABA_{B} receptor (Gabbr1b), each under a CAMKII promoter, and GAD65 under the S5E2 enhancer (see Example 1). This approach was designed to determine if overexpression of each protein would remove a rate limiting step in the surface expression of the functional multimeric receptor or synaptic terminal expression of GABA-synthesizing enzyme, thereby increasing IPSP amplitude.

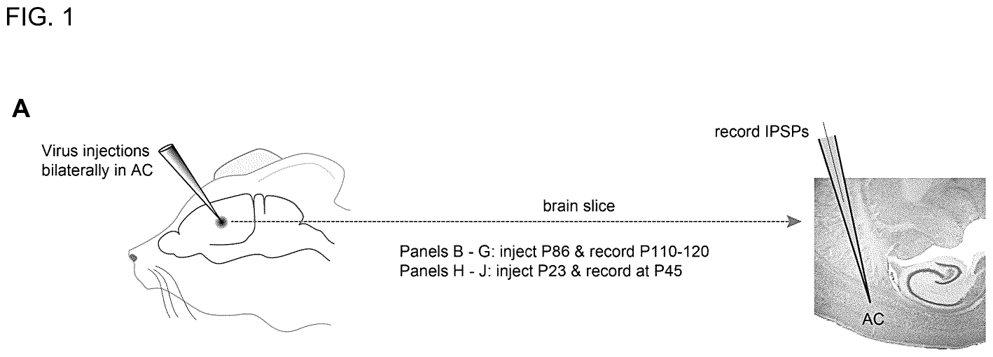

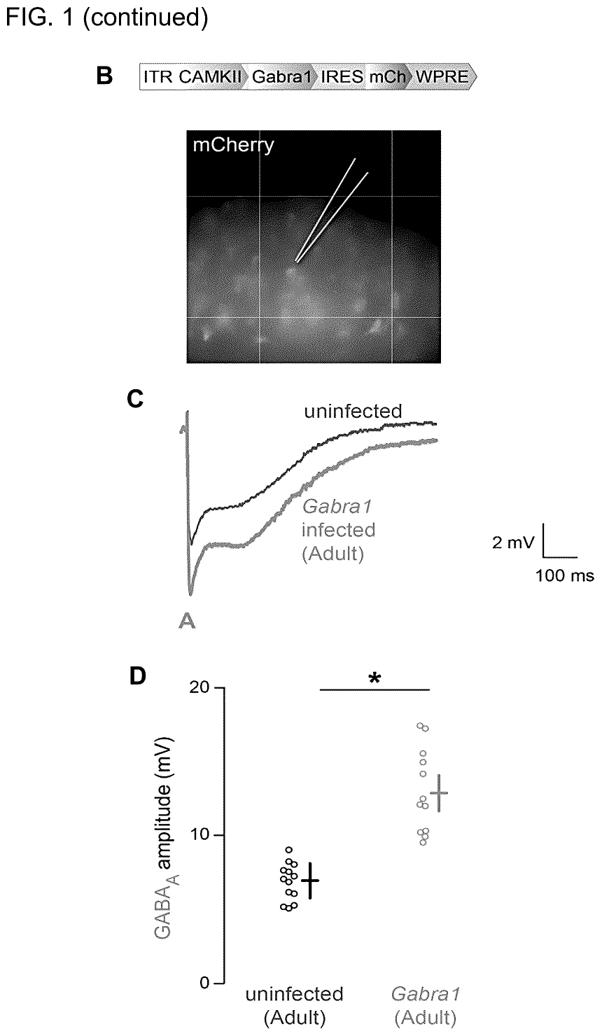

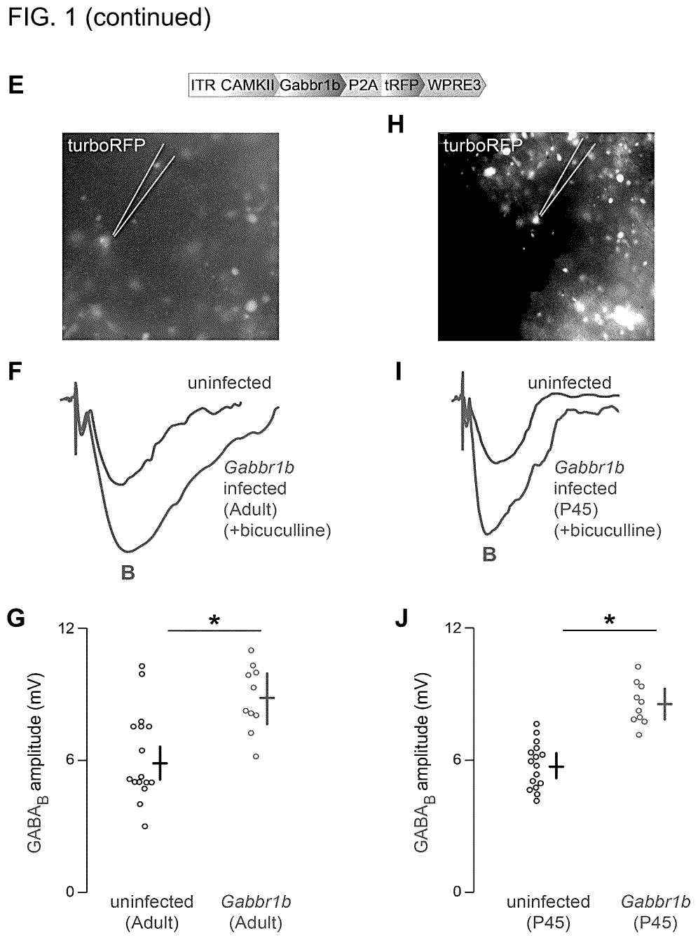

To confirm functional upregulation of cortical inhibitory postsynaptic potentials (IPSPs) following Gabra1 or Gabbr1b expression, we performed whole cell current clamp recordings from pyramidal neurons in auditory cortex brain slices 24-34 days after virus injection into AC at P86 for adult gerbils and 22 days after injection at P23 for young gerbils (FIG. 1, panel A; see Example 1). IPSPs were elicited in response to local electrical stimulation, as described previously (Mowery et al., 2016; Mowery et al., 2019). FIG. 1 panel B shows the vector used to express the Gabra1 subunit under a CaMKII promoter, and a fluorescence image of the reporter molecule, mCherry, as observed during whole cell recordings. FIG. 1 panel C shows representative IPSPs recorded from a Gabra1-infected (orange trace; yellow pipet in panel B) and an uninfected AC neuron (gray trace) at a holding potential (V_{hold}) of −50 mV in the presence of glutamate receptor blockers (20 μM DNQX; 50 μM AP-5). FIG. 1 panel D shows that the peak amplitudes of the short latency IPSP hyperpolarization (putative GABA_{A} component, labeled “A”) was significantly greater for infected neurons (Mean±SEM; infected: 12.8±0.6 mV; uninfected: 6.8±0.6 mV; q=2.05, df=26, p<0.0001). Therefore, the Gabra1 vector increased GABA_{A} receptor-mediated IPSP amplitude in AC pyramidal neurons.

FIG. 1 panel E shows the vector used to express the postsynaptic Gabbr1b subunit (Billinton et al., 1999) under a CamKII promoter, and a fluorescence image of the reporter molecule, turboRFP, as observed during whole cell recordings. FIG. 1 panel F shows representative IPSPs recorded from a Gabbr1b-infected (blue trace; yellow pipet in panel E) and an uninfected AC neuron (gray trace) at a holding potential (V_{hold}) of −50 mV in the presence of glutamate receptor antagonists (20 μM DNQX; 50 μM AP-5) and a GABA_{A} receptor antagonist (10 μM bicuculline). FIG. 1G shows that the peak amplitudes of the long latency IPSP hyperpolarization (putative GABA_{B} component, labeled “B”) was significantly greater for infected neurons (Mean±SEM; infected: 8.7±0.6 mV; uninfected: 5.8±0.4 mV; q=2.03, df=32, p=0.0002). To match the behavioral timeline, the experiment was repeated in animals that received AC injections of the Gabbr1b vector at P23, but recordings were obtained at P45. The effect of vector injection was nearly identical to that observed with older animals (Mean±SEM; infected: 8.5±0.3 mV; uninfected: 5.7=0.3 mV; q=2.06, df=25, p<0.0001). Therefore, the Gabbr1b vector increased GABA_{B} receptor-mediated IPSP amplitude in AC CaMKII-expressing neurons . . .

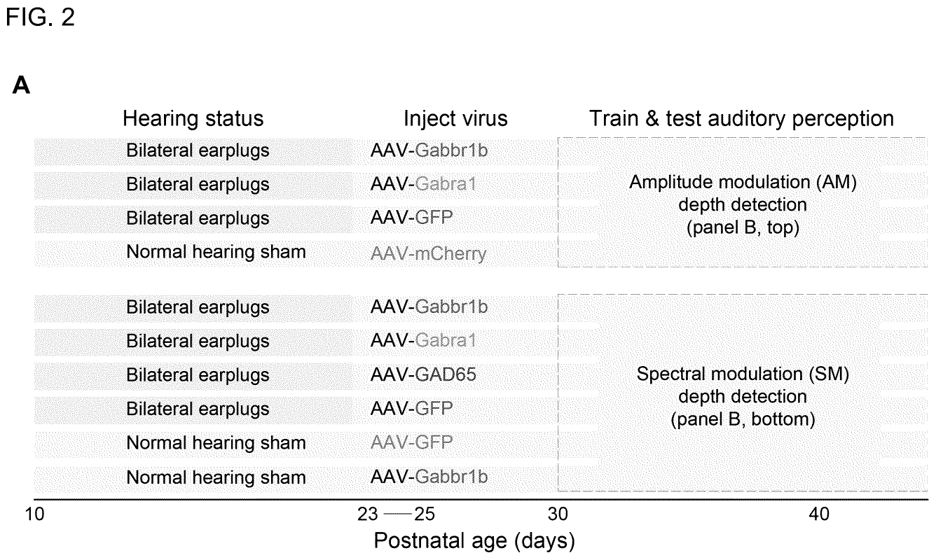

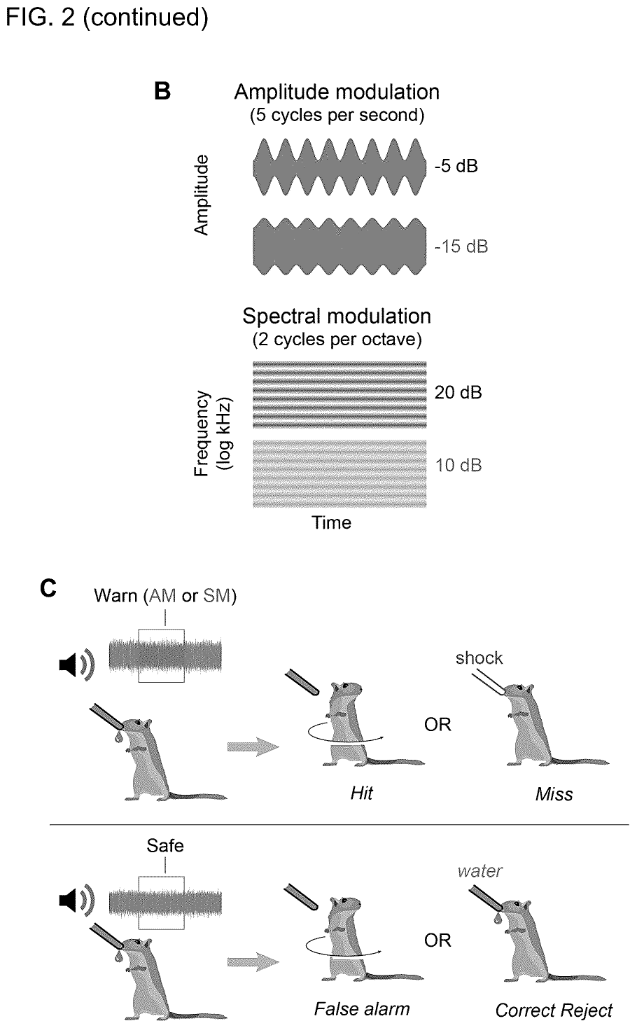

The panels of FIG. 2 outline the full protocol. We induced reversible developmental hearing loss, beginning at ear canal opening (P10) and ending after the auditory critical period (P23) by inserting earplugs (FIG. 2 panel A, orange shading), as described previously (Caras and Sanes, 2015). We injected separate groups of HL-reared animals with the Gabra1 (n=8) or Gabbr1b-expressing vector (n=9), or a GFP control virus (n=9), bilaterally in AC between P23 and P25, after earplug removal. We also included a normal hearing group (n=15; FIG. 2, panel A, gray shading). Following a 7 day recovery period, animals were water restricted and began behavioral training on P30.

Separate groups of animals were tested on AM depth detection (Sarro and Sanes, 2011; Rosen et al., 2012; Caras & Sanes, 2015) or SM depth detection. As described in Example 1, control and HL-reared animals were trained to drink from a lick spout during continuous noise (0.1-20 kHz, 45 dB SPL). For the AM detection task, animals were initially trained to withdraw from the spout when 5 Hz amplitude modulation at 0 dB re: 100% occurred (FIG. 2 panel B, top). For the SM detection task, animals were initially trained to withdraw from the spout when 2 cycles/octave density spectral modulation at 40 dB depth occurred (FIG. 2 panel B, bottom). Procedural learning continued until animals achieved a d′ ≥1.3 with a 0 dB re: 100% depth AM stimuli or 40 dB depth SM stimuli (4-8 days). We then conducted 7 days of psychometric testing as animals' performance gradually improved on the AM or SM task (FIG. 2 panel C). Perceptual thresholds improved as gerbils responded to smaller modulation depths due to perceptual learning (Caras & Sanes, 2017).

We first tested the effect of HL and GABA receptor subunit expression on AM depth detection. FIG. 3 panel A presents representative psychometric functions for two individual animals, and shows that AM detection was superior for the HL-reared gerbil that received bilateral AC injections of a Gabbr1b-expressing vector (HL+Gabbr1b; blue line) as compared to the HL-reared gerbil that received AC bilateral injections of a GFP-expressing vector (HL+GFP; green line). As schematized in FIG. 2 panel A, top, we obtained thresholds for animals in these two groups, as well as HL-reared animals that received a Gabra1-expressing vector (HL+Gabra1), and normal hearing (NH) animals.

FIG. 3 panel B shows thresholds by group over 7 days of psychometric testing. A linear mixed-effects model comparing the effects of training day and virus condition on thresholds, and taking into account individual subject behavior, indicates that viral treatment was a significant factor (F=10.83, p=9.83×10⁻⁷). Therefore, expression of the Gabbr1b subunit restored normal behavioral performance on the AM detection task in HL-reared animals in a manner that was independent of training day.

There was a significant effect of treatment group on day 1 of perceptual testing (one-way ANOVA, p=4.02×10⁻⁵, F=10.46, df=40, FIG. 3 panel C) and day 7 of perceptual testing (one-way ANOVA, p=6.85×10⁻⁸, F=20.07, df=40, FIG. 3 panel D). A post hoc comparison revealed that AM detection thresholds were significantly poorer for transient HL-reared animals that received a control virus (HL+GFP) as compared to normal hearing animals (NH). This was the case both for day 1 (HL+GFP=−5.61±0.99 dB, NH=−10.94±0.77 dB, p=8.35×10⁻⁴) and day 7 of testing (HL+GFP=−8.90±0.91 dB, NH=−14.56±0.70 dB, p=1.00×10⁻⁴). This finding confirms the effect of HL reported previously (Caras and Sanes, 2015; Mowery et al., 2019).

Post hoc comparisons also revealed that GABA_{B} subunit expression, but not GABA_{A} receptor expression could partially restore AM detection thresholds in HL-reared animals. Animals in the HL+Gabbr1b group displayed significantly lower AM detection thresholds than the HL+GFP group, both at day 1 (HL+Gabbr1b=−10.37±0.99 dB, p=0.009, NH=−10.94±0.77 dB, p=8.35×10⁻⁴) and day 7 of perceptual testing (HL+Gabbr1b=−12.47±0.91 dB, p=0.04). In contrast, the HL+Gabra1 group did not different significantly from the HL+GFP group, either at day 1 (HL+GFP=−5.61±0.99 dB, HL+Gabra1=−5.24±1.06 dB, p=99), or day 7 of perceptual testing (HL+GFP=−8.90±0.91 dB, HL+Gabra1=−6.01±0.96 dB, p=0.15).

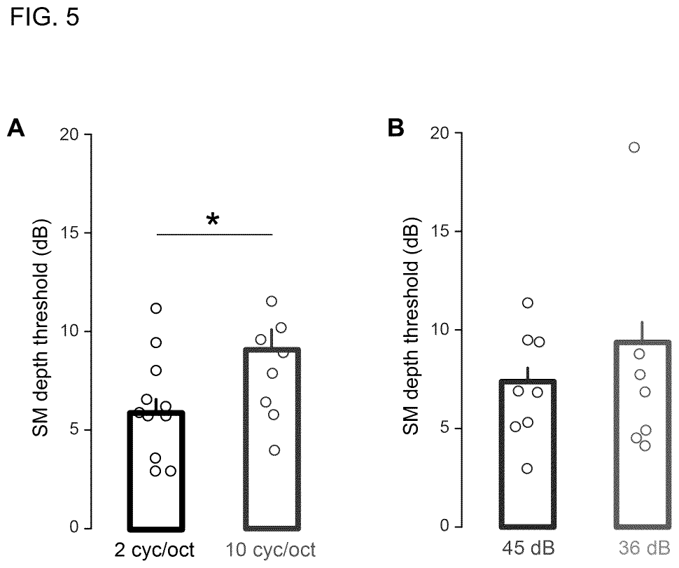

To develop a behavioral test of perception of spectral modulation (SM), we modified the aversive conditioning paradigm such that unmodulated white noise transitioned to spectrally modulated noise on “warn” trials. Similar to the AM detection task, SM stimuli were presented at multiple depths in 3 dB increments to determine each animal's perceptual threshold. Since SM detection has not been assessed previously in gerbils, we first sought to validate two features of this psychometric task. SM is described by a density (cycles/octave) which specifies the peak-to-trough distance of the logarithmic sinusoidal frequency filter used. Humans achieve their best thresholds in the range of 2-4 cycles/octave and thresholds increase at 10 cycles/octave (Eddins and Bero, 2007). We trained gerbils on the same aversive conditioning paradigm used for AM, but with the change cue being a SM stimulus at either 2 or 10 cycles/octave. We found that best thresholds achieved during 10 days of psychometric testing were significantly lower at 2 cycles/octave (5.4±1.43 dB, n=14) than 10 (8.3±2.5 dB; p=0.363, t=−2.148, df=11, unpaired t-test, n=8; FIG. 5). Therefore, all subsequent SM detection psychometric tests used 2 cycles/octave. To confirm that gerbil SM detection is robust to changes in level, as reported for humans (Eddins and Bero, 2007), we alternated between 45 dB SPL and 36 dB SPL during 4 additional days of testing with 2 cycles/octave stimuli (n=8). There was no significant difference in thresholds over each pair of testing days (45 dB days: 9.2±4.0 dB, 36 dB days: 10.66±5.03 dB, p=0.562, t=0.593, df=14, paired t-test). This indicates that gerbil perception of SM stimuli is similar to that displayed by humans.

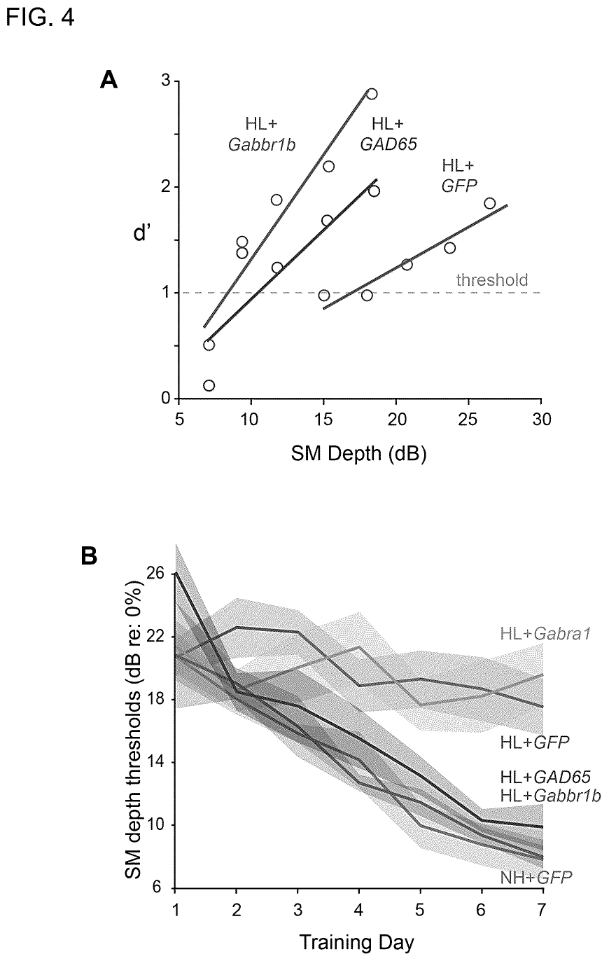

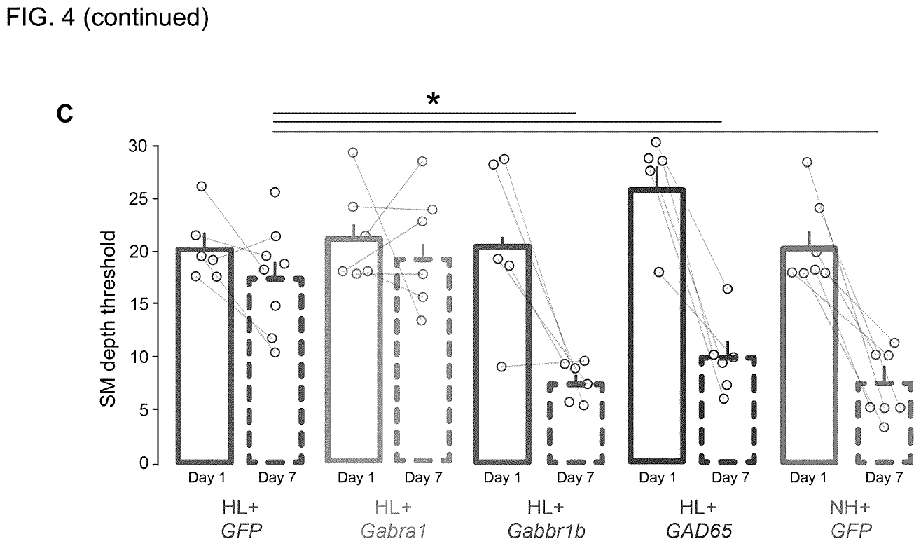

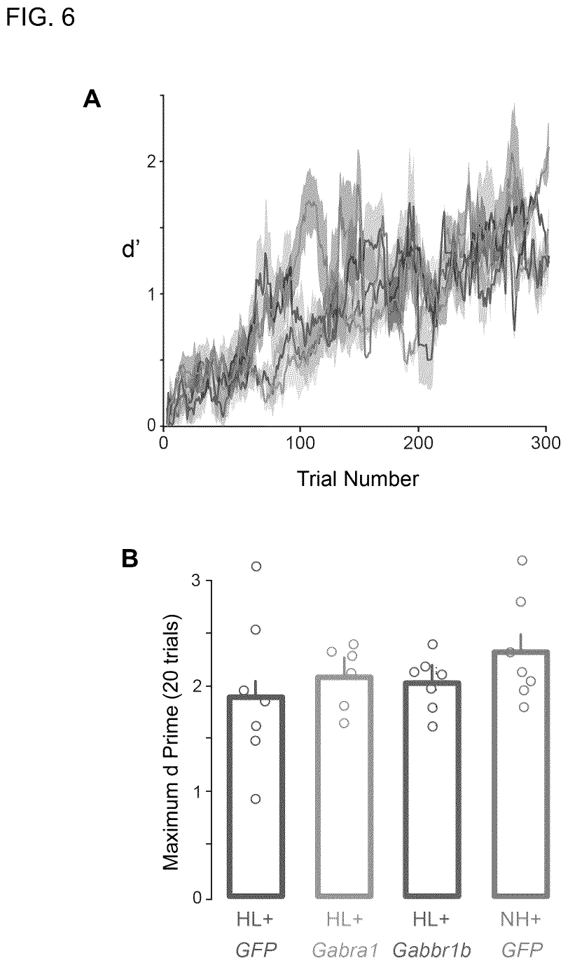

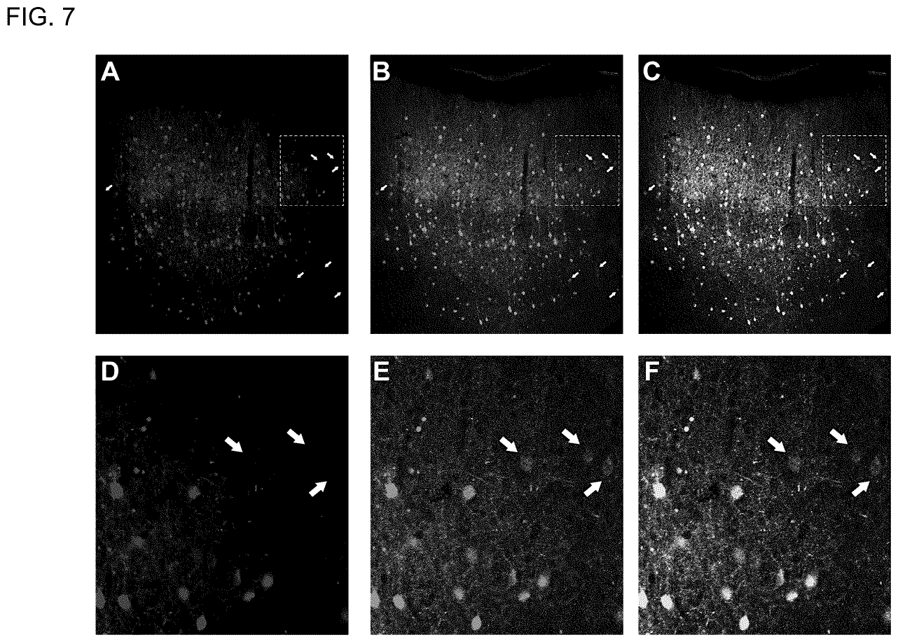

We next tested whether developmental HL would cause an impairment of SM depth detection. As shown in the panels of FIG. 4, HL-reared gerbils displayed significantly poorer SM detection thresholds, as assessed after 7 days of testing (NH+GFP: 7.8±1.6 dB, HL+GFP: 20.7±2.1 dB; statistical comparisons shown below). Therefore, we tested the ability of GABA receptor expression to rescue this second HL-induced perceptual deficit. In order to test a presynaptic source of GABAergic inhibition we developed a viral vector to express the GABA-synthesizing enzyme, GAD65, under the S5E2 enhancer element which targets expression to PV neurons (36). Using immunohistochemistry and confocal imaging we confirmed that turboRFP was expressed in a large majority of neurons that were co-labeled with parvalbumin within the injected area (FIG. 7). All 5 treatment groups reached criterion in a similar number of trials and reached a similar maximum d′ on the SM detection task (FIG. 6), consistent with procedural learning on the AM detection task. FIG. 4 panel A presents representative psychometric functions for individual animals and shows that SM detection was superior for HL-reared gerbils that received bilateral AC injections of a Gabbr1b-expressing vector (HL+Gabbr1b; blue line) or a GAD65-expressing vector (HL+GAD65; purple line) as compared to HL-reared gerbils that received bilateral AC injections of a GFP-expressing vector (HL+GFP; green line). As shown in FIG. 2 panel A, bottom, we obtained thresholds for animals in these three groups, as well as HL-reared animals that received a Gabra1-expressing vector (HL+Gabra1) and normal hearing (NH) animals (HL+GFP, n=8; HL+Gabra1, n=7; HL+GAD65, n=6; HL+Gabbr1b, n=6; NH+GFP, n=7).

FIG. 4 panel B shows group thresholds over all 7 days of training. A linear mixed effects model shows that viral treatment alone is not a significant factor (F=1.623, p=0.169) but the interaction between group and training day was (F=13.652, p=6.389×10⁻¹⁰). Note this statistical analysis included a fifth treatment group discussed below (i.e., GAD65 expression). Therefore, expression of the Gabbr1b subunit in pyramidal neurons restores normal threshold improvement trends on the SM detection task in HL-reared animals.

The SM detection thresholds of all four groups did not differ significantly from one another on day 1 of psychometric testing (HL+GFP: dB=20.66±2.11; HL+Gabra1: dB=21.38±2.11; HL+Gabbr1b: dB=20.83±2.31; NH+GFP: dB=20.77±1.96; ANOVA, p=0.419, F=1.016, df=28, FIG. 4 panel C). However, as shown in FIG. 4 panel D, a significant effect of treatment group emerged by day 7 of testing (one-way ANOVA, p=5.353×10⁻⁶, F=12.396, df=33). NH+GFP and HL+Gabbr1b animals both reached low thresholds of 7.8±1.6 dB and 8.0±1.8 dB, respectively. In contrast, thresholds for HL+GFP (17.5±1.5 dB) and HL+Gabra1 (19.6±1.6 dB) animals improved very little over 7 days of testing. There was no significant difference between day 7 thresholds for HL+Gabbr1b and NH+GFP animals and both were significantly better than HL+GFP (p=0.018, p=0.0009, respectively).

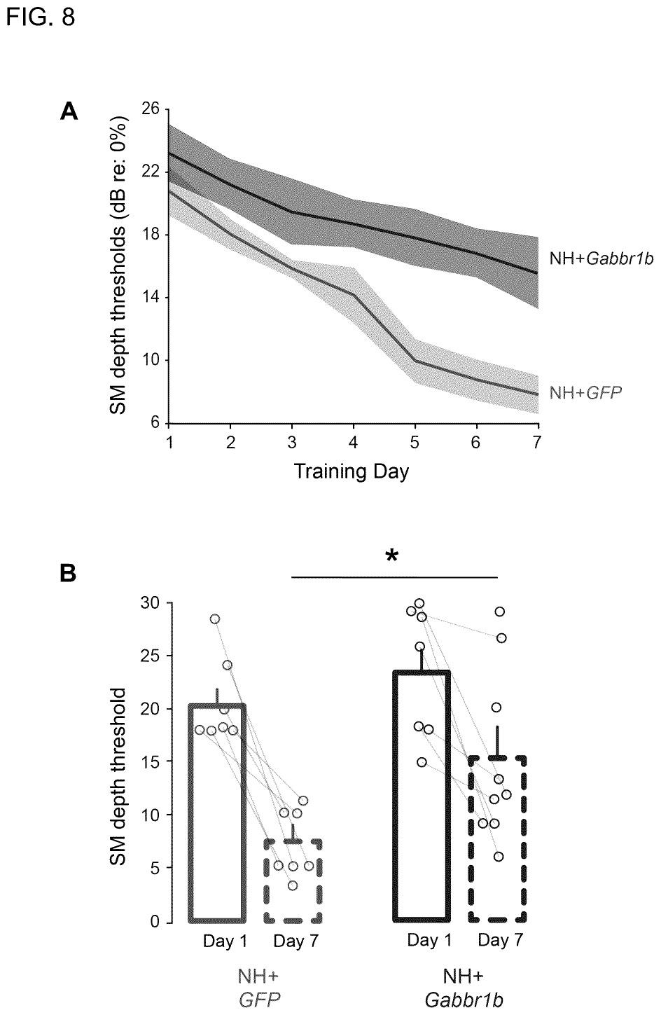

To determine whether the GABAergic synapse vectors improved behavioral discrimination by reversing the inhibitory deficiency, or whether they can improve perception or learning in all animals, we injected the AC with the Gabbr1b vector in a group of sham-treated NH animals (FIG. 8). We found that thresholds on day 1 of testing did not differ from control animals (NH+GFP: 20.77±1.96 dB; NH+Gabbr1b, 23.25±1.85 dB, n=10). However, at day 7 of testing, thresholds were significantly worse than controls (NH+GFP: 7.8±1.6 dB; NH+Gabbr1b, 15.51±2.31 dB; unpaired t-test, p=0.021, t=2.59, df=15).

We interpret these findings to indicate that the vectors improved behavioral performance in HL-reared animals by reversing the inhibitory deficiency.

### Discussion of Examples

Proper regulation of synaptic inhibition is integral to the development and maintenance of sensory processing. Transient or permanent developmental HL that begins during an AC critical period causes a long-lasting reduction of cortical synaptic inhibition that is attributable to the functional loss of both ionotropic GABA_{A} and metabotropic GABA_{B} receptors (Kotak et al., 2005; Takesian et al., 2012; Mowery et al., 2019). This reduction of AC inhibition correlates with impairments in psychometric performance on a range of auditory tasks as well as degraded AC neuron stimulus processing (Rosen et al., 2012; Buran et al., 2014; Caras and Sanes, 2015; Ihlefeld et al., 2016; von Trapp et al., 2017; Yao & Sanes, 2018). To test whether there is a causal relationship between AC inhibition and perceptual thresholds, we upregulated GABA receptor-mediated inhibition through viral expression of the Gabra1 or Gabbr1b subunit genes in putative pyramidal neurons under the CaMKII0.4 promoter, or GAD65 in putative PV neurons under the S5E2 enhancer element. The provided results show that upregulating GABA_{B} receptor-dependent inhibition through expression of the gerbil Gabbr1b subunit gene can rescue two different perceptual deficits, AM and SM detection. Similarly, GAD65 expression can rescue SM detection. In contrast, upregulating GABA_{A} receptor-mediated inhibition through expression of the gerbil Gabra1 subunit gene had no effect on perceptual performance. Therefore, the presently provided results show that the magnitude of AC inhibition is positively correlated with perceptual performance, with postsynaptic GABA_{B} receptors playing a pivotal role.

Amplitude and spectral modulation are discrete features of natural sounds, including speech, and sensitivity to these cues is correlated with speech comprehension (Cazals et al., 1994; Shannon et al., 1995; Singh and Theunissen, 2003; Elliott 2009; Nittrouer et al., 2021). In this disclosure we developed a behavioral paradigm to assess SM detection in gerbils, such that the effects of HL could be compared with a previously characterized percept, AM detection (Rosen et al., 2012; Caras & Sanes, 2015). The transition between modulated and unmodulated noise creates a small artifact during the rise and fall times which is not strictly spectral in nature. The modulation power spectrum of our stimulus, separated into the spectral and temporal domains, showed small but non-zero temporal elements below 5 Hz. We found that rearing conditions and viral treatment had no effect on procedural training times, in agreement with past results with our AM paradigm, although training times were comparatively longer (FIG. 6). Adult humans display depth detection thresholds of ˜2 dB at 2 cycles/octave, with poorer performance at higher spectral densities (Eddins and Bero, 2007). In agreement, we found that gerbils have better thresholds at 2 cycles/octave than 10 cycles/octave and that best thresholds are within 3 dB of human performance at 2 cycles/octave (FIG. 5). Together, this shows agreement with human psychoacoustic studies of SM sensitivity using similar stimuli.

### Interpreting the Pattern of Restored AM and SM Detection Following Gabbr1b Expression

Gabbr1b expression rescued AM and SM detection in HL-reared animals, but with different magnitudes and time courses. For the AM detection task Gabbr1b-treatment improved perceptual performance in HL-reared animals from the first day of testing as compared to GFP-treated controls (FIG. 3). This improved performance was maintained during the 7 days of testing. This outcome is consistent with a rapid improvement in AM stimulus encoding following Gabbr1b expression, but no effect on perceptual learning (i.e., an improvement in detection threshold as a result of practice). In contrast, Gabbr1b expression led to a gradual improvement of SM thresholds during the 7 days of testing, identical to NH animals with GFP expression (FIG. 4). In principle, these differences in the effect of restoring inhibition could relate to differences in the way that AM and SM stimuli are represented in the AC.

Amplitude and spectrally modulated noise are expected to differ in terms of the evoked discharge pattern of AC neurons. AM stimuli are known to produce a strong temporal response that correlates with the AM rate. In contrast, SM stimuli produce a response that is dependent on frequency tuning (e.g., inhibitory sidebands) (Calhoun & Schreiner, 1994, 1998; Atencio & Schreiner, 2010). At the cellular level, one possibility is that feedforward inhibition mediated by Parvalbumin-expressing interneurons tightens the timing of the auditory evoked response in the input Layer 4/5 (Wehr and Zador, 2003; Nocon et al., 2022), which may contribute to perception of amplitude modulation. In contrast, SM stimuli are stationary. Here, intracortical pathways may recruit local interneurons that mediate lateral inhibition, increasing gain in Layer 2/3, thereby improving the detection of energy differences across spectral bands (Kaur et al., 2004; Kaur et al., 2005; Li et. al. 2014).

### Interpreting Restored SM Detection Following GAD65 Expression in PV Neurons

Pyramidal neurons in sensory cortices receive GABAergic input from a variety of interneuron subtypes. PV neurons mediate feedforward inhibition of thalamocortical input and have been implicated in the development and maintenance of mature sensory processing (Xu et al. 2010; He et al. 2014; Cisneros-Franco et al. 2019). Additionally, expression of GAD65 and GAD67 increases throughout early development, while mice lacking GAD65 do not display ocular dominance plasticity even during early development (Guo et al. 1997, Fagiolini et al. 2000). In AC, PV neurons have reduced expression of GAD65 after adult-onset HL, demonstrating that expression levels of the protein are adaptive to sensory input (Miyakawa et al. 2019). Therefore analyzed whether perceptual deficits after developmental HL may be linked to reduced GABA production in PV neurons, in addition to being linked to reduced IPSPs mediated through postsynaptic GABA_{B} receptors. The results showed that over-expressing GAD65 in PV neurons alone had a similar remedial effect to over-expressing GABA_{B} receptors in pyramidal neurons, indicating that both approaches are at least sufficient to restore perception. This provides a strong indication that the perisomatic PV->Pyramidal synapse is a key contributor to developmental deficits in auditory perception and that modulating this synapse in the adult can enhance perception.

### Relationship of GABA Receptor Manipulation to the AC Critical Period

In this disclosure, the manipulations and behavioral assays all occur prior to sexual maturation, a time during which inhibitory functional properties continue to mature (Pinto et al., 2010; Takesian et al., 2012). A large body of research from the developing visual pathway shows that inhibitory synapse development regulates cortical plasticity. Monocular deprivation (MD) leads to reduced cortical activation by the deprived eye during a developmental CP, and experimentally increasing GABAergic transmission can close the critical period prematurely (reviews: Hensch, 2004; Hensch, 2005; Hooks and Chen, 2007). One implication of these observations is that inhibition in adult animals is too strong to permit plasticity. However, manipulations that reduce cortical inhibition in adults can also induce excitatory synaptic plasticity (He et al., 2006; Sale et al., 2007; Fernandez et al., 2007; Harauzov et al., 2010; Cisneros-Franco et al. 2019). Therefore, when inhibitory strength is high, behavioral deficits can be ameliorated by temporarily lowering it. In contrast, the presently provided results suggest that when inhibitory strength is low, as occurs after developmental HL, behavioral deficits can be ameliorated by permanently raising it.

The gerbil AC displays a well characterized critical period (CP) for the effect of HL that closes at P18 (Mowery et al., 2015). When HL is initiated after P18, there is no reduction to AC inhibitory synapse strength (Mowery et al., 2016). In contrast, when HL is initiated before P18, the reduction of AC inhibitory synapse strength persists to adulthood and has been attributed to the functional loss of both GABA_{A} and GABA_{B} receptor-mediated IPSPs (Mowery et al., 2019). Therefore, and without intending to be bound by any particular theory, it is considered that an aspect of this disclosure is that the loss of one or both forms of postsynaptic inhibition is causally related to perceptual deficits that attend developmental HL. Since the virus was injected into AC on P23, the results indicate that the manipulation need not occur during the cortical CP in order to restore normal neural and behavioral function.

We analyzed whether upregulating inhibitory strength through Gabra1 expression (FIG. 1D) would rescue HL-induced deficits on auditory tasks (Fagiolini et al. 2004; Song et al. 2022; Fritzchy et al. 1994). However, we previously reported that systemic treatment with a GABA_{A} al receptor agonist, zolpidem, does not restore AM detection thresholds following developmental HL (see FIG. 2f in Mowery et al., 2019). Two lines of evidence may explain why perceptual performance was rescued only by upregulating GABA_{B} receptor-mediated inhibition. First, GABA_{B} receptor function may directly modulate synaptic plasticity, particularly during development: postsynaptic GABA_{B} receptors can induce inhibitory long-term depression (iLTD) at feedforward inhibitory synapses between PV neurons and pyramidal neurons in input layers of visual cortex during a developmental CP (Wang and Maffei, 2014). This mechanism has been implicated in auditory map remodeling (Vickers et al., 2018) and GABA_{B} receptor agonists enhance ocular dominance plasticity (Cai et al., 2017). Second, postsynaptic GABA_{B} receptors are located extrasynaptically and modulate both the activity of postsynaptic GABA_{A} receptors and NMDA receptor-driven LTP (Komatsu, 1996; Fritschy et al., 1999; Charara et al., 2005; Booker et al., 2013; Tao et al., 2013, Connelly et al. 2013). GABA_{B} receptor activation may also induce BDNF release, thereby inducing the addition of perisomatic GABAergic synapses (Fiorentino et al., 2009). Therefore, although both forms of GABAergic inhibition are reduced by HL, dysregulation of GABA_{B} receptor's modulatory role may have a substantial impact on the acquisition of perceptual skills during development.

The present disclosure demonstrates that restoring inhibition in AC alone was sufficient to restore auditory perception after a developmental insult. Restoring cortical synaptic inhibition may be relevant to a range of developmental disorders. For example, GABA levels in visual cortex are reduced in amblyopia and this is correlated with weaker perceptual suppression by the amblyopic eye (Mukerji et al., 2022). Human patients with Schizophrenia exhibit reduced inhibitory tone as well as reduced expression of GAD65 (Fish et al. 2021). By directly comparing the impact of restoring Gabra1 and Gabbr1b protein expression, the disclosure demonstrates that this effect was only achieved by upregulating GABA_{B} receptor-mediated inhibition. This result is surprising given a long focus on GABA_{A} receptor-mediated inhibition in the field of developmental sensory processing. Additionally, the disclosure shows that upregulating inhibition through PV neurons alone was also sufficient to restore perception. The disclosure supports regulating inhibition for therapies that seek to prevent or reverse behavioral deficits that attend developmental disorders.

This evidence pointing to the role of the GABA_{B} receptor in regulating plasticity also converges on PV neurons, and is supported by the present disclosure. Overexpression of GAD65 in PV neurons should increase overall GABA release at the synaptic cleft, but more specifically than a previously described manipulation using SGRI. This indicates that pre- and post-synaptically upregulating inhibition at perisomatic PV-Pyramidal synapses alone are both sufficient to improve perception and that activation of inhibitory synapses in dendrites (as by Somatostatin-expressing interneurons) is tangential. Upregulation of (AD) 65 in PV neurons is expected to enhance activation of both receptor types in postsynaptic pyramidal neurons. An interpretation from this principle indicates that increased activation of GABA_{A} receptors does not interfere with the beneficial effects of GABA_{B} receptor activation.

While the disclosure has been particularly shown and described with reference to specific embodiments, it should be understood by those having skill in the art that various changes in form and detail may be made therein without departing from the spirit and scope of the present disclosure as disclosed herein.

The following reference listing is not an indication that any reference is material to patentability.
Aizawa, N., & Eggermont, J. J. (2007). Mild noise-induced hearing loss at young age affects temporal modulation transfer functions in adult cat primary auditory cortex. Hearing research, 223 (1-2), 71-82.
Atencio, C. A., & Schreiner, C. E. (2010). Laminar diversity of dynamic sound processing in cat primary auditory cortex. Journal of neurophysiology, 103 (1), 192-205.
Billinton, A., Upton, N., & Bowery, N. G. (1999). GABAB receptor isoforms GBR1a and GBR1b, appear to be associated with pre- and post-synaptic elements respectively in rat and human cerebellum. British journal of pharmacology, 126 (6), 1387-1392.
Booker, S. A., Gross, A., Althof, D., Shigemoto, R., Bettler, B., Frotscher, M., . . . & Vida, I. (2013). Differential GABAB-receptor-mediated effects in perisomatic-and dendrite-targeting parvalbumin interneurons. Journal of Neuroscience, 33 (18), 7961-7974.
Braat, S., & Kooy, R. F. (2015). The GABAA receptor as a therapeutic target for neurodevelopmental disorders. Neuron, 86 (5), 1119-1130.
Buran, B. N., Sarro, E. C., Manno, F. A., Kang, R., Caras, M. L., & Sanes, D. H. (2014). A sensitive period for the impact of hearing loss on auditory perception. Journal of Neuroscience, 34 (6), 2276-2284.
Cai, S., Fischer, Q. S., He, Y., Zhang, L., Liu, H., Daw, N. W., & Yang, Y. (2017). GABAB receptor-dependent bidirectional regulation of critical period ocular dominance plasticity in cats. PLOS One, 12 (6), e0180162.
Calhoun, B. M., & Schreiner, C. E. (1998). Spectral envelope coding in cat primary auditory cortex: linear and non-linear effects of stimulus characteristics. European Journal of Neuroscience, 10 (3), 926-940.
Caras, M. L., & Sanes, D. H. (2015). Sustained perceptual deficits from transient sensory deprivation. Journal of Neuroscience, 35 (30), 10831-10842.
Caras, M. L., & Sanes, D. H. (2017). Top-down modulation of sensory cortex gates perceptual learning. Proceedings of the National Academy of Sciences, 114 (37), 9972-9977.
Caras, M. L., & Sanes, D. H. (2019). Neural variability limits adolescent skill learning. Journal of Neuroscience, 39 (15), 2889-2902.
Cazals, Y., Pelizzone, M., Saudan, O., & Boex, C. (1994). Low-pass filtering in amplitude modulation detection associated with vowel and consonant identification in subjects with cochlear implants. The Journal of the Acoustical Society of America, 96 (4), 2048-2054.
Charara, A., Pare, J. F., Levey, A. I., & Smith, Y. (2005). Synaptic and extrasynaptic GABA-A and GABA-B receptors in the globus pallidus: an electron microscopic immunogold analysis in monkeys. Neuroscience, 131 (4), 917-933.
Choi, J.-H., Yu, N.-K., Baek, G.-C., Bakes, J., Seo, D., Nam, H. J., Baek, S. H., Lim, C.-S., Lee, Y.-S., & Kaang, B.-K. (2014). Optimization of AAV expression cassettes to improve packaging capacity and transgene expression in neurons. Molecular brain, 7 (1), 1-10.
Cisneros-Franco, J. M., & de Villers-Sidani, É. (2019). Reactivation of critical period plasticity in adult auditory cortex through chemogenetic silencing of parvalbumin-positive interneurons. Proceedings of the National Academy of Sciences, 116 (52), 26329-26331.
Connelly, W. M., Fyson, S. J., Errington, A. C., McCafferty, C. P., Cope, D. W., Di Giovanni, G., & Crunelli, V. (2013). GABAB receptors regulate extrasynaptic GABAA receptors. Journal of Neuroscience, 33 (9), 3780-3785.
Dobri, S. G., & Ross, B. (2021). Total GABA level in human auditory cortex is associated with speech-in-noise understanding in older age. Neuroimage, 225, 117474.
Drullman, R. (1995). Temporal envelope and fine structure cues for speech intelligibility. The Journal of the Acoustical Society of America, 97 (1), 585-592.
Edden, R. A., Muthukumaraswamy, S. D., Freeman, T. C., & Singh, K. D. (2009). Orientation discrimination performance is predicted by GABA concentration and gamma oscillation frequency in human primary visual cortex. Journal of Neuroscience, 29 (50), 15721-15726.
Eddins, D. A., & Bero, E. M. (2007). Spectral modulation detection as a function of modulation frequency, carrier bandwidth, and carrier frequency region. The Journal of the Acoustical Society of America, 121 (1), 363-372.
Fagiolini, M., & Hensch, T. K. (2000). Inhibitory threshold for critical-period activation in primary visual cortex. Nature, 404 (6774), 183-186.
Fagiolini, M., Fritschy, J. M., Low, K., Mohler, H., Rudolph, U., & Hensch, T. K. (2004). Specific GABAA circuits for visual cortical plasticity. Science, 303 (5664), 1681-1683.
Fernandez, F., Morishita, W., Zuniga, E., Nguyen, J., Blank, M., Malenka, R. C., & Garner, C. C. (2007). Pharmacotherapy for cognitive impairment in a mouse model of Down syndrome. Nature neuroscience, 10 (4), 411-413.
Fiorentino H, Kuczewski N, Diabira D, Ferrand N, Pangalos M N, Porcher C, Gaiarsa J L (2009) GABA (B) receptor activation triggers BDNF release and promotes the maturation of GABAergic synapses. J Neurosci 29:11650-11661.
Fritschy, J. M., Meskenaite, V., Weinmann, O., Honer, M., Benke, D., & Mohler, H. (1999). GABAB-receptor splice variants GBla and GB1b in rat brain: developmental regulation, cellular distribution and extrasynaptic localization. European Journal of Neuroscience, 11 (3), 761-768.
Fritschy, J. M., Paysan, J., Enna, A., & Mohler, H. (1994). Switch in the expression of rat GABAA-receptor subtypes during postnatal development: an immunohistochemical study. Journal of Neuroscience, 14 (9), 5302-5324.
Fuchs, J. L., & Salazar, E. (1998). Effects of whisker trimming on GABAA receptor binding in the barrel cortex of developing and adult rats. Journal of Comparative Neurology, 395 (2), 209-216.
Gainey, M. A., & Feldman, D. E. (2017). Multiple shared mechanisms for homeostatic plasticity in rodent somatosensory and visual cortex. Philosophical Transactions of the Royal Society B: Biological Sciences, 372 (1715), 20160157.
Gay, J. D., Voytenko, S. V., Galazyuk, A. V., & Rosen, M. J. (2014). Developmental hearing loss impairs signal detection in noise: putative central mechanisms. Frontiers in systems neuroscience, 8, 162.
Gleich, O., Hamann, I., Klump, G. M., Kittel, M., & Strutz, J. (2003). Boosting GABA improves impaired auditory temporal resolution in the gerbil. Neuroreport, 14 (14), 1877-1880.
Green, D. M., & Swets, J. A. (1966). Signal detection theory and psychophysics (Vol. 1, pp. 1969-2012). New York: Wiley.
Guo, Y., Kaplan, I. V., Cooper, N. G., & Mower, G. D. (1997). Expression of two forms of glutamic acid decarboxylase (GAD67 and GAD65) during postnatal development of cat visual cortex. Developmental Brain Research, 103 (2), 127-141.
Han, Y. K., Köver, H., Insanally, M. N., Semerdjian, J. H., & Bao, S. (2007). Early experience impairs perceptual discrimination. Nature neuroscience, 10 (9), 1191-1197.
Harauzov, A., Spolidoro, M., DiCristo, G., De Pasquale, R., Cancedda, L., Pizzorusso, T., . . . & Maffei, L. (2010). Reducing intracortical inhibition in the adult visual cortex promotes ocular dominance plasticity. Journal of Neuroscience, 30 (1), 361-371.
He, H. Y., Hodos, W., & Quinlan, E. M. (2006). Visual deprivation reactivates rapid ocular dominance plasticity in adult visual cortex. Journal of Neuroscience, 26 (11), 2951-2955.
He, L. J., Liu, N., Cheng, T. L., Chen, X. J., Li, Y. D., Shu, Y. S., . . . & Zhang, X. H. (2014). Conditional deletion of Mecp2 in parvalbumin-expressing GABAergic cells results in the absence of critical period plasticity. Nature communications, 5 (1), 5036.
Heffner, H. E., & Heffner, R. S. (1995). Conditioned avoidance. Methods in comparative psychoacoustics, 79-93.
Hensch, T. K. (2004). Critical period regulation. Annu. Rev. Neurosci., 27, 549-579.
Hensch, T. K. (2005). Critical period plasticity in local cortical circuits. Nature Reviews Neuroscience, 6 (11), 877-888.
Hooks, B. M., & Chen, C. (2007). Critical periods in the visual system: changing views for a model of experience-dependent plasticity. Neuron, 56 (2), 312-326.
Horn, D. L., Dudley, D. J., Dedhia, K., Nie, K., Drennan, W. R., Won, J. H., . . . & Werner, L. A. (2017). Effects of age and hearing mechanism on spectral resolution in normal hearing and cochlear-implanted listeners. The Journal of the Acoustical Society of America, 141 (1), 613-623.
Ihlefeld, A., Chen, Y. W., & Sanes, D. H. (2016). Developmental conductive hearing loss reduces modulation masking release. Trends in hearing, 20, 2331216516676255.
Ip, I. B., Emir, U. E., Lunghi, C., Parker, A. J., & Bridge, H. (2021). GABAergic inhibition in the human visual cortex relates to eye dominance. Scientific reports, 11 (1), 1-11.
Iwai, Y., Fagiolini, M., Obata, K., & Hensch, T. K. (2003). Rapid critical period induction by tonic inhibition in visual cortex. Journal of Neuroscience, 23 (17), 6695-6702.
Jiao, Y., Zhang, C., Yanagawa, Y., & Sun, Q. Q. (2006). Major effects of sensory experiences on the neocortical inhibitory circuits. Journal of Neuroscience, 26 (34), 8691-8701.
Kaur, S., Lazar, R., & Metherate, R. (2004). Intracortical pathways determine breadth of subthreshold frequency receptive fields in primary auditory cortex. Journal of neurophysiology, 91 (6), 2551-2567.
Kaur, S., Rose, H. J., Lazar, R., Liang, K., & Metherate, R. (2005). Spectral integration in primary auditory cortex: laminar processing of afferent input, in vivo and in vitro. Neuroscience, 134 (3), 1033-1045.
Kilman, V., Van Rossum, M. C., & Turrigiano, G. G. (2002). Activity deprivation reduces miniature IPSC amplitude by decreasing the number of postsynaptic GABAA receptors clustered at neocortical synapses. Journal of Neuroscience, 22 (4), 1328-1337.
Kim, H., & Bao, S. (2009). Selective increase in representations of sounds repeated at an ethological rate. Journal of Neuroscience, 29 (16), 5163-5169.
Komatsu, Y. (1996). GABAB receptors, monoamine receptors, and postsynaptic inositol trisphosphate-induced Ca2+ release are involved in the induction of long-term potentiation at visual cortical inhibitory synapses. Journal of Neuroscience, 16 (20), 6342-6352.
Kotak, V. C., Takesian, A. E., Mackenzie, P. C., & Sanes, D. H. (2013). Rescue of inhibitory synapse strength following developmental hearing loss. PLOS One, 8 (1), e53438.
Leventhal, A. G., Wang, Y., Pu, M., Zhou, Y., & Ma, Y. (2003). GABA and its agonists improved visual cortical function in senescent monkeys. Science, 300 (5620), 812-815.
Li, L. Y., Ji, X. Y., Liang, F., Li, Y. T., Xiao, Z., Tao, H. W., & Zhang, L. I. (2014). A feedforward inhibitory circuit mediates lateral refinement of sensory representation in upper layer 2/3 of mouse primary auditory cortex. Journal of Neuroscience, 34 (41), 13670-13683.
Maffei, A., Nelson, S. B., & Turrigiano, G. G. (2004). Selective reconfiguration of layer 4 visual cortical circuitry by visual deprivation. Nature neuroscience, 7 (12), 1353-1359.
Merzlyak, E. M., Goedhart, J., Shcherbo, D., Bulina, M. E., Shcheglov, A. S., Fradkov, A. F., . . . & Chudakov, D. M. (2007). Bright monomeric red fluorescent protein with an extended fluorescence lifetime. Nature methods, 4 (7), 555-557.
Miyakawa, A., Wang, W., Cho, S. J., Li, D., Yang, S., & Bao, S. (2019). Tinnitus correlates with downregulation of cortical glutamate decarboxylase 65 expression but not auditory cortical map reorganization. Journal of Neuroscience, 39 (50), 9989-10001.
Möhler, H., Fritschy, J. M., Crestani, F., Hensch, T., & Rudolph, U. (2004). Specific GABAA circuits in brain development and therapy. Biochemical pharmacology, 68 (8), 1685-1690.
Mowery, T. M., Caras, M. L., Hassan, S. I., Wang, D. J., Dimidschstein, J., Fishell, G., & Sanes, D. H. (2019). Preserving inhibition during developmental hearing loss rescues auditory learning and perception. Journal of Neuroscience, 39 (42), 8347-8361.
Mowery, T. M., Kotak, V. C., & Sanes, D. H. (2015). Transient hearing loss within a critical period causes persistent changes to cellular properties in adult auditory cortex. Cerebral cortex, 25 (8), 2083-2094.
Mowery, T. M., Kotak, V. C., & Sanes, D. H. (2016). The onset of visual experience gates auditory cortex critical periods. Nature communications, 7 (1), 1-11.
Mukerji, A., Byrne, K. N., Yang, E., Levi, D. M., & Silver, M. A. (2022). Visual cortical y-aminobutyric acid and perceptual suppression in amblyopia. Frontiers in Human Neuroscience, 16.
Nittrouer, S., Lowenstein, J. H., & Sinex, D. G. (2021). The contribution of spectral processing to the acquisition of phonological sensitivity by adolescent cochlear implant users and normal-hearing controls. The Journal of the Acoustical Society of America, 150 (3), 2116-2130.
Nocon, J. C., Gritton, H. J., James, N. M., Han, X., & Sen, K. (2022). Parvalbumin neurons, temporal coding, and cortical noise in complex scene analysis. bioRxiv, 2021-09.
Ozmeral, E. J., Eddins, A. C., & Eddins, D. A. (2018). How do age and hearing loss impact spectral envelope perception? Journal of Speech, Language, and Hearing Research, 61 (9), 2376-2385.
Pinto, J. G., Hornby, K. R., Jones, D. G., & Murphy, K. M. (2010). Developmental changes in GABAergic mechanisms in human visual cortex across the lifespan. Frontiers in cellular neuroscience, 16.
Polley, D. B., Thompson, J. H., & Guo, W. (2013). Brief hearing loss disrupts binaural integration during two early critical periods of auditory cortex development. Nature communications, 4 (1), 1-13.
Rosen, M. J., Sarro, E. C., Kelly, J. B., & Sanes, D. H. (2012). Diminished behavioral and neural sensitivity to sound modulation is associated with moderate developmental hearing loss. PLOS ONE 7 (7): e41514.
Sale, A., Berardi, N., Spolidoro, M., Baroncelli, L., & Maffei, L. (2010). GABAergic inhibition in visual cortical plasticity. Frontiers in cellular neuroscience, 10.
Sanes, D. H., & Kotak, V. C. (2011). Developmental plasticity of auditory cortical inhibitory synapses. Hearing research, 279 (1-2), 140-148.
Sarro, E. C., & Sanes, D. H. (2011). The cost and benefit of juvenile training on adult perceptual skill. Journal of Neuroscience, 31 (14), 5383-5391.
Sarro, E. C., Kotak, V. C., Sanes, D. H., & Aoki, C. (2008). Hearing loss alters the subcellular distribution of presynaptic GAD and postsynaptic GABAA receptors in the auditory cortex. Cerebral Cortex, 18 (12), 2855-2867.
Schreiner, C. E., & Calhoun, B. M. (1994). Spectral envelope coding in cat primary auditory cortex: properties of ripple transfer functions. Aud Neurosci, 1 (1), 39-62.
Shannon, R. V., Zeng, F. G., Kamath, V., Wygonski, J., & Ekelid, M. (1995). Speech recognition with primarily temporal cues. Science, 270 (5234), 303-304.
Singh, N. C., & Theunissen, F. E. (2003). Modulation spectra of natural sounds and ethological theories of auditory processing. The Journal of the Acoustical Society of America, 114 (6), 3394-3411.
Song, Y. J., Xing, B., Barbour, A. J., Zhou, C., & Jensen, F. E. (2022). Dysregulation of GABAA receptor-mediated neurotransmission during the auditory cortex critical period in the fragile X syndrome mouse model. Cerebral Cortex, 32 (1), 197-215.
Szymczak, A. L., & Vignali, D. A. (2005). Development of 2A peptide-based strategies in the design of multicistronic vectors. Expert opinion on biological therapy, 5 (5), 627-638.
Takesian, A. E., Kotak, V. C., & Sanes, D. H. (2012). Age-dependent effect of hearing loss on cortical inhibitory synapse function. Journal of neurophysiology, 107 (3), 937-947.
Tao, W., Higgs, M. H., Spain, W. J., & Ransom, C. B. (2013). Postsynaptic GABAB receptors enhance extrasynaptic GABAA receptor function in dentate gyrus granule cells. Journal of Neuroscience, 33 (9), 3738-3743.
Vickers, E. D., Clark, C., Osypenko, D., Fratzl, A., Kochubey, O., Bettler, B., & Schneggenburger, R. (2018). Parvalbumin-interneuron output synapses show spike-timing-dependent plasticity that contributes to auditory map remodeling. Neuron, 99 (4), 720-735.
von Trapp, G., Aloni, I., Young, S., Semple, M. N., & Sanes, D. H. (2017). Developmental hearing loss impedes auditory task learning and performance in gerbils. Hearing research, 347, 3-10.
Vormstein-Schneider, D., Lin, J. D., Pelkey, K. A., Chittajallu, R., Guo, B., Arias-Garcia, M. A., . . . & Dimidschstein, J. (2020). Viral manipulation of functionally distinct interneurons in mice, non-human primates and humans. Nature neuroscience, 23 (12), 1629-1636.
Wang, L., & Maffei, A. (2014). Inhibitory plasticity dictates the sign of plasticity at excitatory synapses. Journal of Neuroscience, 34 (4), 1083-1093.
Wehr, M., & Zador, A. M. (2003). Balanced inhibition underlies tuning and sharpens spike timing in auditory cortex. Nature, 426 (6965), 442-446.
Wichmann, F. A., & Hill, N. J. (2001). The psychometric function: I. Fitting, sampling, and goodness of fit. Perception & psychophysics, 63 (8), 1293-1313.
Wichmann, F. A., & Hill, N. J. (2001). The psychometric function: II. Bootstrap-based confidence intervals and sampling. Perception & psychophysics, 63 (8), 1314-1329.
Xu, J., Yu, L., Cai, R., Zhang, J., & Sun, X. (2010). Early continuous white noise exposure alters auditory spatial sensitivity and expression of GAD65 and GABAA receptor subunits in rat auditory cortex. Cerebral Cortex, 20 (4), 804-812.
Yao, J. D., & Sanes, D. H. (2018). Developmental deprivation-induced perceptual and cortical processing deficits in awake-behaving animals. Elife, 7, e33891.
Zeng, F. G., Nie, K., Stickney, G. S., Kong, Y. Y., Vongphoe, M., Bhargave, A., . . . & Cao, K. (2005). Speech recognition with amplitude and frequency modulations. Proceedings of the National Academy of Sciences, 102 (7), 2293-2298.
Zhang, L. I., Bao, S., & Merzenich, M. M. (2001). Persistent and specific influences of early acoustic environments on primary auditory cortex. Nature neuroscience, 4 (11), 1123-1130.
Zorio, D. A., Monsma, S., Sanes, D. H., Golding, N. L., Rubel, E. W., & Wang, Y. (2019). De novo sequencing and initial annotation of the Mongolian gerbil (Meriones unguiculatus) genome. Genomics, 111 (3), 441-449.
Zufferey, R., Donello, J. E., Trono, D., & Hope, T. J. (1999). Woodchuck hepatitis virus posttranscriptional regulatory element enhances expression of transgenes delivered by retroviral vectors. Journal of virology, 73 (4), 2886-2892.

What is claimed is:

1. A recombinant polynucleotide for use in reducing central nervous system sequelae of hearing loss, or improving hearing in an individual in need thereof, the recombinant polynucleotide comprising a genetic element that is operably linked to a sequence encoding a 65-kD isoform of glutamate decarboxylase (GAD65).

2. The recombinant polynucleotide of claim 1, wherein the genetic element selectively drives expression of the GAD65 protein in neuronal cells, said neuronal cells optionally being parvalbumin-positive interneurons (PV neurons).

3. The recombinant polynucleotide of claim 2, wherein genetic element comprises a S5E2 enhancer.

4. The recombinant polynucleotide of claim 3, wherein the recombinant polynucleotide is comprised by an expression vector.

5. The recombinant polynucleotide of claim 4, wherein the expression vector comprises a viral vector.

6. The recombinant polynucleotide of claim 5, wherein the expression vector comprises an adenoviral expression vector.

7. Viral particles comprising the recombinant polynucleotide of claim 6.

8. A method comprising administering to an individual in need thereof an effective amount of viral particles of claim 7 such that the sequelae of hearing loss in the individual is inhibited, or hearing of the individual is improved.

9. The method of claim 8, wherein the GAD65 protein is selectively expressed in neuronal cells present in the auditory cortex of the individual, and wherein the neuronal cells are PV neurons.

10. The method of claim 8, wherein the hearing of the individual is improved.

11. The method of claim 9, wherein the hearing of the individual is improved.

12. A method comprising introducing into neuronal cells a recombinant polynucleotide of claim 1, such that the neuronal cells express the GAD65 protein, wherein the expression of the GAD65 protein is driven by an S5E2 enhancer.

13. The method of claim 12, wherein the genetic element selectively drives expression of the GAD65 protein in neuronal cells, said neuronal cells optionally being parvalbumin-positive interneurons (PV neurons).

14. The method of claim 13, wherein genetic element comprises an S5E2 enhancer.

15. The method of claim 14, wherein the recombinant polynucleotide is comprised by an expression vector.

16. The method of claim 15, wherein the expression vector comprises a viral vector.

17. The method of claim 16, wherein the expression vector comprises an adenoviral expression vector.
